(19) United States
(12) Patent Application Publication
(10) Pub. No.: US 2012/0330093 A1
(43) Pub. Date: Dec. 27, 2012

(54) MEDICAL PRODUCT AND PRODUCTION THEREOF

(75) Inventors: Erich Odermatt, Schaffhausen (CH); Christine Weis, Sant Cugat del Valles (ES); Tania Nieves, Terrassa (Barcelona) (ES); Erhard Mueller, Stuttgart (DE); Hans-Gerd Schmees, Wannweil (DE)

(73) Assignees: ITV DENKENDORF PRODUKTSERVICE GMBH, Denkendorf (DE); AESCULAP AG, Tuttlingen/Donau (DE)

(21) Appl. No.: 13/451,929

(22) Filed: Apr. 20, 2012

(30) Foreign Application Priority Data
Apr. 21, 2011 (DE) 10 2011 007 844.4

(51) Int. Cl. A61B 17/03; B26D 1/00; A61F 2/04

(52) U.S. Cl. 600/30; 606/151; 83/13

(57) ABSTRACT

A medical product for use in treating/managing hernias, prolapses, urinary incontinence and/or dyspareunia in humans and/or animals including at least one textile fabric having a textile ground structure and thread structures which protrude from the trextile ground structure, wherein at least some of the thread structures are interloped with the textile ground structure.

### RELATED APPLICATION

This application claims priority of German Patent Application No. 10 2011 007 844.4, filed Apr. 21, 2011, herein incorporated by reference.

### TECHNICAL FIELD

This disclosure relates to a medical product in the form of a textile fabric, to a medical product in the form of a spacer fabric and also to corresponding production processes.

### BACKGROUND

Self-retaining implants are generally implants that do not need additional fixing aids such as, for example, suture materials, adhesives, clips or the like for retention/anchoring in the body of a human or animal patient. Self-retaining implants have self-anchorable sub-structures in the form of barbs, pile threads, thread loops and the like, for example.

Implants having such sub-structures that at least augment anchoring in the body are already known from, for example:
DE 19 912 648 A1 describes a sheetlike implant for use in surgery comprising a flexible fabric, wherein one side of the fabric may include textured yarns, floats and/or velour loops to improve the cellular ingrowth behaviour.
DE 10 2007 063 214 A1 relates to a hernia implant in the form of a first textile fabric coated uniformly and sealingly, and a second textile fabric connected to the first fabric in a conjoint connecting plane and which is free of any coating. There may be places on the implant for body cells to get behind, for example, textured yarns, floats, velour loops and/or tufts.
WO 2009/071998 A2 discloses an implant to prevent or treating stomal hernias which may have fastening means such as, for example, loops and barbs on one side for fastening to the abdominal wall.
WO 01/81667 A1 relates to a prosthetic knit for medical or surgical use wherein a ply of monofil threads on one side of the knit has protruding picot hairs.
Polymeric meshes with anchoring hooks useful particularly for the surgical management of laparoceles form part of the subject matter of DE 20 2004 017 304 U1.
A wound closure device with barbs is known from US 2002/0077661 A1.
EP 2 229 918 A1 discloses a self-retaining implant with barbs. This implant is likewise particularly suitable for hernia management.

Difficulties can arise with these kinds of implants where the sub-structures provided for retention or self-retention are not firmly integrated in the implant structure. In those cases, there is a risk that the sub-structures will become dislodged out of the implant under tensile stress, directly impairing the structural integrity and hence the (self-)retaining properties of the implant.

It could therefore be helpful to provide a medical product having (self-)retaining properties which avoids known disadvantages and, more particularly, provides secure retention/anchoring even under the influence of tensile-load forces as may typically arise in the body of a human or animal patient.

### SUMMARY

We provide a medical product for use in treating/managing hernias, prolapses, urinary incontinence and/or dyspareunia in humans and/or animals comprising at least one textile fabric having a textile ground structure and thread structures which protrude from the textile ground structure, wherein at least some of the thread structures are interloped with the textile ground structure.

We also provide a medical product as a spacer fabric with two mutually opposite textile ground structures spaced apart from each other via spacer threads, wherein at least some of the spacer threads are interloped with at least one of the two textile ground structures.

We further provide a process for producing one or more medical products, wherein spacer threads of a spacer fabric which space apart two mutually opposite textile ground structures from each other are severed by cutting through to obtain two textile fabrics having a textile ground structure and thread structures protruding from the textile ground structure.

### BRIEF DESCRIPTION OF THE DRAWINGS

FIGS. 1a to e schematically show examples of products in the form of a textile fabric.

FIGS. 2a to f schematically show products in the form of a spacer fabric.

FIGS. 3a to d schematically show products in the form of two textile fabrics laid on top of each other.

FIG. 4 shows the lapping diagram for Production Example 1.

FIG. 5 shows the lapping diagram for Production Example 2.

FIG. 6 shows the lapping diagram for Production Example 3.

### DETAILED DESCRIPTION

It will be appreciated that the following description is intended to refer to specific examples of structure selected for illustration in the drawings and is not intended to define or limit the disclosure, other than in the appended claims.

We provide a medical product comprising at least one textile fabric having a textile ground structure and thread structures which protrude from the textile ground structure, wherein at least some of the thread structures are interlooped with the textile ground structure, i.e., at least some of the thread structures are tied into the textile ground structure in the form of at least one interloop.

In other words, the thread structures—at least some of them—are at one end interlooped with the textile ground structure, while their free ends protrude from the textile ground structure.

The expression “at least one textile fabric” shall be understood as meaning in general one textile fabric or a multiplicity of textile fabrics, especially two, three or more textile fabrics.

Preferably, the medical product is a textile fabric having a textile ground structure and thread structures which protrude from the textile ground structure with at least some of the thread structures being interlooped with the textile ground structure, i.e., at least some of the thread structures being tied into the textile ground structure in the form of at least one interloop.

The medical product is advantageous because the thread structures interlooped with the textile ground structure cannot be pulled out of the textile ground structure on exposure to tensile loads typically arising in the body of a patient. As a result, the thread structures, which in functional respects can be conceived of as retaining or anchoring structures, are able to contribute to a secure/reliable retention, especially self-retention, of the product in human or animal tissue. There is accordingly no risk of the thread structures becoming lost under tensile stress and thereby possibly causing an undesired dislocation of the product in the body of a patient.

Interlooping the thread structures with the textile ground structure preferably also has the effect that the stiffness of the medical product is increased, which is advantageous especially with regard to the unfurlability of the medical product, for example, after delivery from a suitable delivering instrument such as a trocar in particular.

The expression “in the form of at least one interloop” shall be understood as meaning in general “in the form of one interloop and/or in the form of a multiplicity of interloops, for example, in the form of two, three and/or four interloops.”

Preferably, all thread structures are interlooped with the textile ground structure, i.e., tied into the textile ground structure in the form of at least one interloop.

Also, the thread structures may be tied pairwise into the textile ground structure in the form of at least one interloop.

The at least one interloop may be configured as pillar, tricot, atlas, cord, filet and/or velvet stitch.

Further, threads, preferably all threads, of the at least one textile fabric, especially of the textile ground structure and/or the thread structures, are free from anchoring structures, especially sticking-out anchoring structures such as barbs, for example.

More particularly, threads, preferably all threads of the at least one textile fabric, especially of the textile ground structure and/or the thread structures, have no incisions on the thread surface, especially no anchoring structures configured as incisions on the thread surface. This has the advantage with regard to the thread structures that the thread diameter has no incision-caused taperings which could possibly impair the mechanical stability and especially the (self-)retaining properties of the medical product.

The thread structures are preferably threads, especially severed, preferably cut, cut-off or cut-through, threads.

Preferably, the thread structures are configured as tuft/pile threads, especially as velour threads, velvet threads, plush threads or combinations thereof, or in the manner of tuft/pile threads, especially in the manner of velour threads, velvet threads, plush threads or combinations thereof.

The thread structures may have a cut area at their free ends.

The thread structures may be configured cylindrically or essentially cylindrically at their free ends, in which case the cylindrical basal areas at the free ends are preferably each configured as cut area.

The thread structures may project from the textile ground structure perpendicularly or essentially perpendicularly. The expression “essentially perpendicularly” is here to be understood as meaning that the thread structures can optionally project away from the textile ground structure at an angle below about 90°, especially at an angle about 45 to about 90°, preferably at an angle about 70 to about 90° and more preferably at an angle about 80 to about 90°. An angle<90° may be advantageous with regard to lower tissue irritation when fixing the product in the body of a patient.

In the case of thread structures projecting perpendicularly from the textile ground structure, the extent of tissue irritation can be controlled with particular advantage through the length with which the thread structures project from the textile ground structure.

The thread structures may further have a straight-line or essentially straight-line body and have a head at each of their free ends. The head is preferably wider than the body of the thread structures. For example, the head may be widened relative to the body in the manner of a mushroom head. A head that is broader than the body may be advantageous in tissue fixation.

The thread structures particularly preferably and more particularly on at least one side of the at least one textile fabric are severed, preferably cut-through, spacer or connecting threads of a spacer fabric, especially of a knitted spacer fabric, in particular a loop-formingly knitted spacer fabric, preferably of a spacer fabric configured as purl fabric or rib fabric, preferably rib fabric.

A purl fabric is generally a textile fabric, especially a spacer fabric, where the technical back and the technical face of the fabric, especially the outer technical back and the outer technical face of the fabric, each show reverse interloops.

A rib fabric is generally a textile fabric, especially a spacer fabric, where the technical back and the technical face of the fabric, especially the outer technical back and the outer technical face of the fabric, each show face interloops.

A plain-knit fabric is generally a textile fabric, especially a spacer fabric, whose technical face, especially exterior technical face, shows face interloops and whose technical back, especially exterior technical back, shows reverse interloops.

Each interloop or interlooped stitch of an interlooped fabric such as a formed-loop knit, for example, generally consists of a head, two legs and two feet. Where the legs transition into the feet there are two points of contact with the preceding interloop which are known as intermeshing points. When the heads and feet of the interloops point up and the legs correspondingly down at these intermeshing points, reverse interloops and the technical back of an interlooped fabric are concerned. If, by contrast, the feet are down and the legs up, face interloops and the technical face of an interlooped fabric are concerned. With regard to the definition of reverse and face interloops and of the technical back and the technical face, reference may also be made here to standard works in textile technology.

A particular advantage of a spacer fabric configured as rib fabric is that the binding of the spacer or connecting threads in the form of at least one interloop in the ground structure is freely chooseable.

Particularly preferably, the at least one textile fabric includes at least two thread plies, especially two or three thread plies, preferably just two thread plies.

The at least one textile fabric may include two thread plies, of which one thread ply forms the textile ground structure and the other thread ply forms the thread structures.

Alternatively, the at least one textile fabric may include three thread plies of which two thread plies form the textile ground structure and the third thread ply forms the thread structures.

The thread structures are preferably only configured on one side (one-sidedly), especially just on one surface side of the textile ground structure. Preferably, additional thread structures are formed on an opposite side, especially on an opposite surface side of the textile ground structure and preferably likewise protrude from the textile ground structure. The expression “additional thread structures,” hereinafter also called “additionally present thread structures” or “additionally provided thread structures,” is to be understood as meaning thread structures which, in addition to our thread structures, may protrude from what is generally an opposite side of the textile ground structure.

The thread structures and the optionally additionally present thread structures may be configured the same or differently. More particularly, the optionally additionally provided thread structures may also be interlooped with the textile ground structure. However, there can be differences, for example, with regard to the thread material, the thread diameter, the thread length, especially the tuft or pile length, the thread linear density, the thread structure and the like. Further differences may reside in a different pattern, a different arrangement and/or in a different density with which the thread structures and the optionally additionally present thread structures can be configured on the surface of the textile ground structure.

The additional thread structures may be thread loops, more particularly selected from the group consisting of pile loops, velour loops, velvet loops, terry loops, plush loops, floats and combinations thereof, and/or be tuft/pile threads, more particularly selected from the group consisting of velour threads, velvet threads, plush threads, terry threads and combinations thereof.

Preferably, the at least one textile fabric has thread structures, especially in the form of severed spacer or connecting threads of a spacer fabric on one side and thread loops, especially in the form of velour and/or terry loops, on an opposite side.

Alternatively preferably, the at least one textile fabric has the thread structures, especially in the form of severed spacer or connecting threads of a spacer fabric on one side and sticking-out threads in the form of tuft or pile threads on an opposite side.

The thread structures and/or the optionally additionally present thread structures may be monofil, pseudomonofil and/or multifil, especially braided or twisted. However, it is particularly preferable for the thread structures and/or the optionally additionally present thread structures to have a monofil, pseudomonofil and/or mono-filament-like configuration. As a result, the thread structures and/or the optionally additionally present thread structures have a higher flexural stiffness, which is advantageous especially with regard to the retention of the product in the human or animal body.

The thread structures and/or the optionally additionally present thread structures may have a diameter of about 50 to about 300 μm, especially about 80 to about 250 μm and preferably about 80 to about 160 μm.

The thread structures and/or the optionally additionally present thread structures may further be configured on the textile ground structure in a density, especially tuft density, of about 10 to about 330 per cm², especially about 19 to about 225 per cm² and preferably about 20 to about 50 per cm².

The thread structures and/or the optionally additionally present thread structures may further have a length, especially a tuft length, of about 0.1 to about 5 mm, especially of about 0.5 to about 4 mm and preferably of about 0.5 to about 2.5 mm.

The medical product, especially the at least one textile fabric, preferably the textile ground structure, the thread structures and/or the optionally additionally present thread structures, may be configured to be absorbable, partially absorbable and/or non-absorbable.

It is preferable for the medical product and especially for the at least one textile fabric to have at least one absorbable fraction. This makes it possible to reduce the proportion of material remaining permanently in the body of a patient, resulting in effect in a reduced input of exogenous material into the body of a patient.

Further, the medical product, especially the at least one textile fabric, preferably the textile ground structure, the thread structures and/or the optionally additionally present thread structures, may be formed of an absorbable material, especially of an absorbable polymer. The polymer may be a synthetic/manufactured polymer and/or a so-called “biopolymer,” i.e., a naturally occurring polymer such as, for example, a protein and/or a polysaccharide. It is preferable for the absorbable polymer to be a polyester, especially a polyhydroxyalkanoate.

The absorbable polymer may be selected from the group consisting of poly-lactide, polyglycolide, poly-g-caprolactone, poly-para-dioxanone, polytrimethylene car-bonate, polyhydroxybutyrate, poly-3-hydroxybutyrate, poly-4-hydroxybutyrate, polyhydroxyvalerate, copolymers, for example, terpolymers thereof and combinations thereof.

Copolymers are polymers composed of two or more, for example, three different monomeric units. Copolymers may be random copolymers or block copolymers.

Alternatively or in combination to the preceding examples, the absorbable polymer may be selected from the group consisting of collagen, gelatin, elastin, reticulin, laminin, fibronectin, silk, especially spider's silk, albumin, fibrin, fibrinogen, starch, amylose, amylopectin, dextran, chitosan, cellulose, alkylcelluloses, especially methylcellulose, carboxyalkylcelluloses, especially carboxymethylcellulose, hyaluronic acid, heparin, heparan sulphate, chondroitin sulphate, dermatan sulphate, keratan sulphate, salts thereof and combinations thereof.

One example of a preferred absorbable copolymer is a copolymer comprising glycolide and lactide, especially in a ratio of 95 wt %:5 wt % to 5 wt %:95 wt % and preferably of 90 wt %:10 wt % to 80 wt %:20 wt %. A further example of a preferred copolymer is a copolymer comprising L-lactide and D,L-lactide, especially in a ratio of 95 wt %:5 wt % to 5 wt %:95 wt %, preferably 80 wt %:20 wt % to 20 wt %:80 wt % and more preferably 70 wt %:30 wt % to 30 wt %:70 wt %. A preferred terpolymer is a terpolymer, especially block terpolymer, comprising glycolide, trimethylene carbonate and g-caprolactone. A terpolymer of this type is commercially available under the designation Monosyn®.

The medical product, especially the at least one textile fabric, preferably the textile ground structure, the thread structures and/or the optionally additionally present thread structures, may be formed of a non-absorbable material, especially of a non-absorbable polymer, preferably selected from the group consisting of polyolefins, polyesters, polyamides, polyurethanes, copolymers, for example, terpolymers thereof and combinations thereof.

The non-absorbable polymer may be selected from the group consisting of polypropylene, polyethylene, high density polyethylene (HDPE), low density polyethylene (LDPE), high molecular weight polyethylene (HMWPE), ultrahigh molecular weight polyethylene (UHMWPE), polyvinylidene dichloride, polyvinylidene difluoride, polytetrafluoroethylene, polyhexafluoropropylene, polytetrafluoropropylene, polyethylene terephthalate, polypropylene terephthalate, polybutylene terephthalate, polyetheretherketone (PEEK), nylon, nylon-6, nylon-6,6, nylon-6,12, polyurethane, copolymers, for example terpolymers, thereof and combinations thereof.

Particularly preferably, the thread structures and/or the optionally additionally present thread structures are configured to be absorbable, while the textile ground structure is configured to be partially absorbable or non-absorbable, preferably non-absorbable.

An advantage of thread structures configured to be absorbable is that absorption events in vivo are generally associated with inflammatory reactions which stimulate the formation of new connective tissue. This usually means a stimulation of connective tissue in the region of the thread structures which enables improved retention in the body of a patient to be achieved for the product.

It may further be provided for the at least one textile fabric, especially the textile ground structure, the thread structures and/or the optionally additionally present thread structures, to be formed of a mixture of materials described above, especially polymers.

Advantageously, the thread structures and/or the optionally additionally present thread structures may be formed of, or be coated with, a material, especially polymer, which is swellable in fluids, especially bodily fluids such as, for example, tissue fluids and/or blood, and/or is tissue adhesive. The material is preferably present as hydrogel. Suitable materials may be selected, for example, from the group consisting of polyvinyl alcohol, polyvinylpyrrolidone, polypropylene glycol, polypropylene oxide, polytetramethylene oxide, proteins, polysaccharides, especially mucopolysaccharides, copolymers, for example, terpolymers thereof, salts thereof and combinations thereof.

Proteins may be fiber-shaped, especially extracellular, proteins and/or globular proteins. Preferred proteins are selected from the group consisting of collagen, gelatin, elastin, reticulin, laminin, fibronectin, albumin, fibrin, fibrinogen, salts thereof and combinations thereof.

Suitable polysaccharides may be selected from the group consisting of starch, amylose, amylopectin, dextran, chitosan, cellulose, alkylcelluloses, especially methylcellulose, carboxyalkylcelluloses, especially carboxymethylcellulose, hyaluronic acid, heparin, heparan sulphate, chondroitin sulphate, dermatan sulphate, keratan sulphate, salts thereof and combinations thereof.

The textile ground structure may further be formed of a different material/polymer or a different material/polymer mixture than the thread structures and/or the optionally additionally present thread structures. With regard to possible materials/polymers, full reference is made to the description hereinabove.

However, it is similarly preferable for all sub-structures of the at least one textile fabric, especially the textile ground structure, the thread structures and the optionally additionally present thread structures, to be formed of the same poly-mer/material or the same material/polymer mixture. With regard to possible materials/polymers, full reference is likewise made to the description hereinabove.

The thread structures and/or the optionally additionally present thread structures may have a core-sheath construction. For example, the core may be formed of a non-absorbable material, especially non-absorbable polymer, and the sheath of an absorbable material, especially an absorbable polymer. The choice of material for core and sheath can be used to specifically adjust/control the absorbability of the thread structures and/or of the optionally additionally present thread structures in particular.

The thread structures and/or the optionally additionally present thread structures may in particular be bicomponent threads. The expression “bicomponent threads” is to be understood as meaning threads formed of two different materials, especially two different polymers.

Further advantageously, sub-structures of the at least one textile fabric, especially the textile ground structure, the thread structures and/or the optionally additionally present thread structures, may absorb at different rates, i.e., have different absorption times. It may be provided, for example, that the thread structures and/or the optionally additionally present thread structures have a shorter absorption time than the textile ground structure.

The thread structures and/or the optionally additionally present thread structures may be arranged in the form of a pattern, for example, in the form of a circular, oval-shaped, triangular, quadrangular, rectangular, square, pentangular and/or hexangular pattern, on the surface of the textile ground structure.

In addition, the thread structures and/or the optionally additionally present thread structures may be arranged on the surface of the textile ground structure in an arrangement selected from the group consisting of row-shaped arrangement, staggered arrangement, overlapping arrangement, spiral- or helical-shaped arrangement, serpentine-shaped arrangement, meander-shaped arrangement, adventitious or random arrangement and combinations thereof.

The textile ground structure typically has a textile structure based on threads, preferably polymer threads. With regard to the material/polymer of which the threads may be formed, reference is made to the materials/polymers mentioned herein.

The textile ground structure is preferably configured as interlooped fabric, especially as formed-loop knit or drawn-loop knit, preferably as formed-loop knit, especially as warp knit.

The textile ground structure is further preferably configured to be porous.

More particularly, the textile ground structure may have interloops, especially pores, having a clear size/diameter of about 100 to about 10 000 μm, especially of about 250 to about 8000 μm and preferably of about 500 to about 5000 μm.

The textile ground structure may have interloops configured as pillar, tricot, cloth, atlas, filet and/or velvet stitches.

It is particularly preferable for the textile ground structure to be configured as a mesh, preferably as a loop-formingly knitted mesh and especially as a warp-knitted mesh.

The at least one textile fabric may have a basis weight of about 40 to about 200 g/m², especially about 50 to about 100 g/m².

It is preferable for the at least one textile fabric to have a basis weight of about 25 to about 100 g/m² and especially about 25 to about 75 g/m² after absorption of an absorbable fraction.

The thread structures may protrude from the textile ground structure on one side of the textile fabric only, while the opposite side may have a coating, especially a uniform and preferably sealing coating. The coating may be formed, for example, of collagen, gelatin, polyvinyl alcohol, salts thereof or the like. It is preferable for the coating to have anti-adhesive properties.

The medical product may comprise two textile fabrics, while at least one of the two textile fabrics, especially only one of the two textile fabrics, has a textile ground structure with thread structures which protrude from the textile ground structure, while at least some of the thread structures are interlooped with the textile ground structure.

Preferably, both the textile fabrics each have a textile ground structure and thread structures which protrude from the textile ground structure, while at least some of the thread structures are interlooped with the textile ground structure.

The two textile fabrics, especially their textile ground structures, thread structures and/or their optionally additionally present thread structures, may be configured to be the same or different. A differing configuration of the textile fabrics may be based, for example, on different textile structures, intermeshings, thread materials, thread diameters, thread linear densities, thread structures, interloop/pore shapes, interloop/pore sizes, basis weights, absorbability/nonabsorbability, absorption times and/or other properties.

The textile ground structure of one textile fabric may be present, for example, as interlooped fabric, especially as formed-loop knit or drawn-loop knit, while the textile ground structure of the other textile fabric is a non-woven structure, especially a fibrous nonwoven web or a laid scrim.

However, it is particularly preferable for the two textile fabrics to be configured the same, especially their textile ground structures each being configured as interlooped fabric, preferably as formed-loop knit, and especially as a loop-formingly knitted mesh.

The two textile fabrics may be in connection with each other. More particularly, the two textile fabrics may be interlinked.

Preferably, the two textile fabrics are arranged/laid one above the other. It is particularly preferable for at least some of the thread structures, especially all the thread structures of the bottom textile fabric to project through interloops, especially pores in the top textile fabric. The bottom textile fabric in this arrangement and more particularly thread structures protruding from its ground structure may be bonded, more particularly adhered, thermofixed and/or fused, more particularly ultrasonically fused, to the textile ground structure of the top textile fabric. It is preferable for the bottom textile fabric to be configured to be absorbable and for the top textile fabric to be configured to be non-absorbable. The top textile fabric may be formed of polypropylene, for example. The examples described in this section have the advantage that they make it possible to increase the thread structures on one side of the product provided to retain, especially self-retain, the product in the body of a patient. The other side, by contrast, may, for example, have a coating, preferably a smooth and preferably sealing and, more particularly, anti-adhesive, coating and/or additional thread structures.

With regard to further features and advantages whereby the medical product may comprise two textile fabrics, full reference is made to the description hereinabove and hereinbelow.

The at least one textile fabric may in principle be configured as double-backed fabric, single-faced fabric or double-faced fabric. It is preferable for the at least one textile fabric to be embodied as single-faced fabric, preferably as interlooped single-faced fabric and more particularly as loop-formingly plain-knitted fabric.

The medical product, especially the at least one textile fabric and preferably the textile ground structure, the thread structures and/or the optionally additionally present thread structures, may be provided with additives, especially active agents such as, for example, active medical/pharmaceutical agents, active biological agents and/or other active agents. The active agents may be selected, for example, from the group consisting of active antimicrobial, especially antibiotic, agents such as, for example, silver, active disinfecting agents, active anti-inflammatory agents, active analgesic agents, active agents that promote cellular growth, active agents that stimulate cellular differentiation, active agents that promote cellular migration, active agents that promote cellular adhesion, salts thereof and combinations thereof.

The medical product is preferably configured as a surgical implant, especially as a self-retaining implant. Self-retention on the part of our implants is obtainable, for example, by the implants being laid onto the tissue surfaces to be treated/managed and pressed down onto the tissue surfaces which forces the thread structures into the tissue and ensures a generally sufficient degree of retention of the implant in the body of a patient.

The medical product is preferably configured as an implant for treatment/management of hernias, thoracal defects, prolapses, urinary incontinence and/or dyspareunia in humans and/or animals.

It is particularly preferable for the medical product to be a surgical mesh, especially a self-retaining mesh.

The medical product may be selected from the group consisting of hernia mesh, prolapse mesh, incontinence mesh, dyspareunia mesh, stoma mesh and thorax mesh.

We further provide a medical product comprising a spacer fabric with at least two, especially two, opposite textile ground structures spaced apart from each other via spacer threads (connecting threads), wherein at least some of the spacer threads are interlooped with at least one of the two textile ground structures, especially with both textile ground structures, i.e., at least some of the spacer threads being tied in the form of at least one interloop in at least one of the two textile ground structures and especially into both textile ground structures.

Preferably, all spacer threads are interlooped with at least one of the two textile ground structures and especially with both textile ground structures, i.e., tied in the form of at least one interloop in at least one of the two textile ground structures and especially into both textile ground structures.

Alternatively, the spacer threads are merely interlooped with one of the two textile ground structures, and not interlooped with the other textile ground structure and more particularly are connected to the other textile ground structure in some other way. For example, the spacer threads can be adhered, thermofixed and/or fused to the other textile ground structure.

Threads, preferably all threads, of the spacer fabric, especially of the textile ground structures, and/or the spacer threads, may be free from anchoring structures, especially sticking-out anchoring structures, for example, barbs.

More particularly, threads, preferably all threads, of the spacer fabric, especially of the textile ground structures, and/or the spacer threads, have no incisions on the thread surface, especially no anchoring structures configured as incisions on the thread surface.

The spacer threads may in principle be configured as monofil, pseudomonofil and/or multifil, especially braided or twisted. However, it is particularly preferable for the spacer threads to be configured as monofil, pseudomonofil and/or monofilament-like.

Advantageously, the two textile ground structures may have projecting thread structures on that side which face away from the spacer threads. In an alternative, only one of the two textile ground structures has projecting thread structures on that side which faces away from the spacer threads. The thread structures preferably serve to retain, especially self-retain, the medical product or spacer fabric. The thread structures may be configured as thread loops, more particularly selected from the group consisting of pile loops, velour loops, velvet loops, terry loops, plush loops, floats and combinations thereof, and/or as tuft/pile threads, more particularly selected from the group consisting of velour threads, velvet threads, plush threads, terry threads and combinations thereof.

The spacer threads may be interposed with thread loops, more particularly selected from the group consisting of velour loops, velvet loops, terry loops, plush loops, floats and combinations thereof. More particularly, one thread loop may be configured between two spacer threads at a time.

The two textile ground structures of the spacer fabric may in principle be configured the same or differently. When the textile ground structures are configured differently, the differing configuration may be based, for example, on different intermeshings, thread materials, thread diameters, thread linear densities, thread structures, textile structures, interloop/pore shapes, interloop/pore sizes, basis weights, absorbability/nonabsorbability, absorption times and/or other properties.

In principle, the spacer fabric, especially at least one of the two textile ground structures and preferably both textile ground structures, and/or the spacer threads, may be configured to be absorbable, partially absorbable and/or non-absorbable.

Preferably, the spacer threads are configured to be absorbable.

At least one of the two textile ground structures, especially only one of the two textile ground structures, may be configured to be absorbable.

It may further be preferable for one of the two textile ground structures to be configured to be absorbable and for the other textile ground structure to be configured to be non-absorbable.

Preferably, the spacer threads and one of the two textile ground structures are configured to be absorbable, while the second textile ground structure is preferably configured to be non-absorbable.

The medical product or spacer fabric, especially at least one of the two textile ground structures and preferably both textile ground structures, and/or the spacer threads, may be formed of an absorbable material, especially an absorbable polymer, for example, a synthetic/manufactured polymer and/or a biopolymer, preferably a polyhydroxyalkanoate.

The absorbable polymer may be selected from the group consisting of polylactide, polyglycolide, poly-ε-caprolactone, poly-para-dioxanone, polytrimethylene carbonate, polyhydroxybutyrate, poly-3-hydroxybutyrate, poly-4-hydroxybutyrate, polyhydroxyvalerate, copolymers, for example, terpolymers thereof and combinations thereof.

Alternatively or in combination to the preceding examples, the absorbable polymer may be selected from the group consisting of collagen, gelatin, elastin, reticulin, laminin, fibronectin, silk, especially spider's silk, albumin, fibrin, fibrinogen, starch, amylose, amylopectin, dextran, chitosan, cellulose, alkylcelluloses, especially methylcellulose, carboxyalkylcelluloses, especially carboxymethylcellulose, hyaluronic acid, heparin, heparan sulphate, chondroitin sulphate, dermatan sulphate, keratan sulphate, salts thereof and combinations thereof.

The medical product or the spacer fabric, in particular at least one of the two textile ground structures and preferably both textile ground structures, and/or the spacer threads, may be formed of a non-absorbable material, especially of a non-absorbable polymer, preferably selected from the group consisting of polyolefins, polyesters, polyamides, polyurethanes, polyetheretherketones, copolymers, for example, terpolymers thereof and combinations thereof.

The non-absorbable polymer may be selected from the group consisting of polypropylene, polyethylene, high density polyethylene (HDPE), low density polyethylene (LDPE), high molecular weight polyethylene (HMWPE), ultrahigh molecular weight polyethylene (UHMWPE), polyvinylidene dichloride, polyvinylidene difluoride, polytetrafluoroethylene, polyhexafluoropropylene, polytetrafluoropropylene, polyethylene terephthalate, polypropylene terephthalate, polybutylene terephthalate, polyetheretherketone (PEEK), nylon, nylon-6, nylon-6,6, nylon-6,12, polyurethane, copolymers, for example, terpolymers thereof and combinations thereof.

At least one of the two textile ground structures, especially both textile ground structures, may be configured as interlooped fabric such as, for example, formed-loop knit or drawn-loop knit or as non-woven structure such as, for example, fibrous nonwoven web or laid scrim.

It is preferable for at least one of the two textile ground structures, especially both textile ground structures, to be embodied as interlooped fabric, preferably as formed-loop knit and especially as warp knit.

It is particularly preferable for at least one of the two textile ground structures, especially both textile ground structures, to be present as mesh, preferably as loop-formingly knitted mesh and especially as warp-knitted mesh.

The spacer threads may be interlooped with a loop-formingly knitted and preferably mesh-configured textile ground structure and connected to an opposite, preferably fibrous nonwoven web type, textile ground structure in a manner other than interlooping, especially by adhering, thermofixing and/or fusing.

The spacer fabric may comprise more than two, for example, three opposite textile ground structures. The ground structures are preferably spaced apart from each other via spacer threads which are at least partly interlooped with the ground structures. With regard to further features and advantages concerning the spacer fabric, especially the textile ground structures and/or the spacer threads, the description hereinabove is referenced in full.

The spacer fabric itself may in principle be a double-backed fabric, single-faced fabric or double-faced fabric. The spacer fabric is preferably a double-faced fabric, especially an interlooped double-faced fabric and preferably a loop-formingly knitted double-faced or rib fabric.

The medical product or the spacer fabric, especially at least one of the two textile ground structures, preferably both textile ground structures, and/or the spacer threads, may further include additives, especially active agents. With regard to the possible additives, especially active agents, the description hereinabove is referenced in full.

The medical product is preferably a surgical implant, especially a self-retaining implant.

With regard to further features and advantages of the medical product or spacer fabric, especially of the two textile ground structures, and/or the spacer threads, the observations made above are referenced in full.

We further provide a process for producing one or more, especially two, medical products, wherein spacer threads of a spacer fabric which space apart two mutually opposite textile ground structures from each other are severed, preferably cut through, to obtain two textile fabrics having a textile ground structure and thread structures protruding from the textile ground structure. The two textile fabrics each may serve as a medical product.

The two textile ground structures may be reconnected to each other, if desired. Any suitable physical and/or chemical method may be contemplated for this in principle.

Advantageously, the two textile fabrics obtained may be arranged/laid on top of each other, for example, such that at least some of the thread structures, especially all thread structures of the bottom textile fabric project through interloops, especially pores, in the top textile fabric. To connect the two textile fabrics together, the bottom textile fabric and more particularly thread structures protruding from its ground structure, may be adhered, thermofixed and/or fused, especially ultrasonically fused, to the ground structure of the top textile fabric.

Preferably, the spacer threads are interlooped with at least one of the two textile ground structures, preferably with both textile ground structures, to produce the spacer fabric.

It is particularly preferable for the spacer threads to be tied into at least one of the two textile ground structures, preferably into both textile ground structures, in the form of at least one interloop.

Preferably, the spacer fabric is produced by formed-loop knitting.

The spacer fabric is produced using a formed-loop knitting machine and especially a three-dimensional formed-loop knitting machine The formed-loop knitting machine preferably has at least two and especially exactly two formed-loop knitting needle beds. Each formed-loop knitting needle bed typically has formed-loop knitting needles and also a knock-over comb bar. The distance between the knock-over comb bar and the formed-loop knitting needle bed can be used to determine the distance between the two textile ground structures in the final spacer fabric.

By using a three-dimensional formed-loop knitting machine, the length and/or density of the spacer threads and hence of the thread structures resulting therefrom after severing the spacer or connecting threads can be widely varied, for example. Using a three-dimensional formed-loop knitting machine further allows the production of spacer fabrics having two differently configured textile ground structures. For instance, a three-dimensional formed-loop knitting machine enables one-step production of two textile ground structures which are mutually spaced apart via spacer threads and which differ especially in respect of the textile type of intermeshing or lapping, the thread material, the thread diameter, the thread linear density, the thread structure, the interloop/pore shape (for example, hexagonal structures, diamond structures, lattice structures and the like), the interloop/pore size, the basis weight, the absorbability/nonabsorbability, the absorption time and/or other properties.

Depending on the materials used, the textile fabrics obtained after severing the spacer or connecting threads may, as already mentioned, also be reconnected again physically and/or chemically.

Furthermore, textile ground structures having large repeats can be produced using a three-dimensional formed-loop knitting machine.

In principle, the spacer fabric can be produced as double-faced fabric, single-faced fabric or double-backed fabric. The spacer fabric is preferably produced as double-faced fabric. Producing the spacer fabric as double-faced fabric has the particular advantage that the threading of the spacer threads is freely chooseable. It is possible, for example, to use six fully threaded guide bars and six empty guide bars per repeat of the spacer fabric or of the two textile ground structures. Alternatively, two fully threaded guide bars and two empty guide bars can be used per repeat. Alternatively, one fully threaded guide bar and one empty guide bar can be used per repeat. It is otherwise likewise possible for fully threaded guide bars, for example, six fully threaded guide bars, to be used exclusively per repeat.

The spacer threads may in principle be severed during the production of the spacer fabric or thereafter.

Preferably, the spacer threads are severed at or in a formed-loop knitting machine. Useful cutting instruments for this can more particularly be a structural component of the formed-loop knitting machine, for example. This enables precise and more particularly controlled severing of spacer threads.

It is particularly preferable for the spacer threads to be severed immediately after the interlooping (interloop-forming process), preferably at or in a three-dimensional formed-loop knitting machine.

The spacer threads may be severed, preferably cut through, mechanically, thermally, for example, using a hot wire, ultrasound or a laser. When the spacer threads are severed thermally, especially with a hot wire, the thread structures resulting therefrom generally have head-shaped, more particularly mushroom head-shaped, widenings at their free ends.

Thread loops may be produced on at least one of the two textile ground structures and especially on both textile ground structures on that side which faces away from the spacer threads or thread structures. The thread loops may be formed during or after the production of spacer fabric. Severing the spacer threads provides with particular advantage one textile fabric or two textile fabrics which each have one side with thread structures protruding from the textile ground structure in the form of severed spacer threads and an opposite side with thread loops. The thread loops may be produced via alternating thread insertions using needles and/or pins. The thread loops may be further selected from the group consisting of pile loops, velour loops, velvet loops, terry loops, plush loops, floats and combinations thereof.

The thread loops may be severed. This is a particularly advantageous way of obtaining two textile fabrics which have thread structures in the form of severed spacer threads on one side and severed/opened thread loops or a cut pile (cut pile threads) on an opposite side.

Thread loops can be produced between the spacer threads during production of the spacer fabric. More particularly, one or optionally more than one thread loop can be produced between any two adjacent spacer threads. Such thread loops are readily obtainable during the production process of the spacer fabric using a formed-loop knitting machine and especially a three-dimensional formed-loop knitting machine.

The advantage of the process described herein is chiefly that one production step provides two medical products each in the form of a textile fabric having thread structures protruding from its textile ground structure, especially in the manner of a cut pile, without any need for subsequent processing steps such as the severing of thread loops, for example.

By contrast, conventional processes for producing products comprising a cut loop pile initially involve producing thread loops out of a textile ground structure which have to be severed in a subsequent step to form a cut loop pile.

A further advantage of our process is that the two textile fabrics obtained can be joined together to form a novel implant, especially a composite implant. In this regard, full reference is made to the description heretofore.

Interlooping the spacer threads with the textile ground structures ensures that the thread structures resulting therefrom after severing the spacer or connecting threads can no longer be pulled out of the textile ground structures under tensile load, since the interloops would pull tight in such case. This preserves the structural integrity of the medical products obtainable by following the process described above, at least during the initial post-operative healing phase in the case of partially or completely absorbable products.

Further advantages are that the position and/or length of the spacer threads are freely settable. Better setting is also possible of the angles with which the thread structures resulting (after severing) from the spacer or connecting threads can protrude from the textile ground structures. Furthermore, the textile ground structures may further be configured differently. Moreover, interloop/pore size adjustability is better. Finally, the use of adhesive in regions of the textile ground structures or of threads can also be avoided.

With regard to further features and advantages of the process, especially of the two textile ground structures and/or the spacer threads, the description heretofore is referenced in full.

We still further provide a process for producing a medical product comprising:
a) producing, preferably by formed-loop knitting, a textile fabric having a textile ground structure and thread loops which preferably protrude from the textile ground structure, and
b) severing, preferably by cutting through, the thread loops to form thread structures which protrude out of the textile ground structure.

The thread loops are preferably produced using a pile guide bar. Alternatively, however, the thread loops can also be produced via alternating thread insertions using needles and/or pins.

Advantageously, step a) may be carried out using a formed-loop knitting machine and more particularly a warp-knitting machine.

For this the formed-loop knitting machine preferably has a formed-loop knitting needle bed and a pile guide bar.

The thread loops can be severed, preferably cut through, mechanically, thermally, for example, with a hot wire, ultrasound or a laser.

With regard to further features and advantages of the process, particularly in respect of the textile fabric and/or the thread loops, the description hereinabove is referenced in full.

We also provide a process for producing a medical product in the form of a spacer fabric wherein spacer threads (connecting threads) are interlooped with at least one of two textile ground structures which form opposite each other, or with at least one of two ready-produced textile ground structures opposite each other.

Preferably, the spacer threads are interlooped with two textile ground structures forming opposite each other or with two ready-formed textile ground structures opposite each other.

The spacer threads are preferably tied into the two textile ground structures in the form of at least one interloop.

The spacer fabric is preferably produced using a formed-loop knitting machine, especially a three-dimensional formed-loop knitting machine, preferably with two formed-loop knitting needle beds.

With regard to further features and advantages of the process, especially in respect of the textile ground structures and/or the spacer or connecting threads, the description hereinabove is referenced in full.

Further features and advantages of our medical products and processes will become apparent from the following examples, figure descriptions and figures. Individual features herein can each be actualized on their own or in combination with each or one another. The examples described hereinbelow merely serve to assist understanding and must not be construed as in any way limiting.

### Production Example 1

The medical product was produced in two stages proceeding from polypropylene threads in that, in a first step, a three-dimensional formed-loop knitting machine was used to produce a three-dimensional formed-loop knit having two opposite loop-formingly knitted, mesh-shaped ground structures and spacer threads which space the ground structures apart from each other and, in a second step, the spacer threads were severed.

Severing the spacer threads took place immediately following interloop formation at/in the formed-loop knitting machine and the two severed loop-formingly knitted ground structures were wound up separately.

The three-dimensional formed-loop knit was produced using a three-dimensional formed-loop knitting machine having two formed-loop knitting needle beds (front and back needle beds) and three guide bars (guide bars 7, 4 and 2).

One of the two textile ground structures was produced in atlas lapping (two courses and closed) using guide bar 7 (only one guide bar on the front bed/full threading):
(2-3/2-2/1-2/1-1/1-0/1-1/2-1/2-2)×2.

The second textile ground structure was produced in atlas lapping (two courses and closed) using guide bar 2 (only one guide bar on the back bed/full threading):
(2-2/1-2/1-1/1-0/1-1/2-1/2-2/2-3)×2.

Guide bar 4 was used to connect the two textile ground structures via spacer threads by binding the spacer threads into every second interloop of the ground structures (being formed). The interloops were produced on the front and back needle beds (threaded one in/one out):
(0-1/0-0/0-0/1-0/1-0/0-0/0-0/0-1/0-1/0-0/0-0/1-0/1-0/0-0/0-0/0-1//.

Guide bars 7 and 4 were used for the front of the three-dimensional formed-loop knit and guide bars 2 and 4 for the back.

The spacer threads were severed using a hot wire to obtain two medical products each having a textile ground structure and thread structures protruding from the textile ground structure. The medical products had altogether two thread plies, one of which formed the textile ground structure and the other, the thread structures protruding from the textile ground structure.

### Production Example 2

The medical product was produced in two stages proceeding from polypropylene threads in that, in a first step, a three-dimensional formed-loop knitting machine was used to produce a three-dimensional formed-loop knit having two opposite loop-formingly knitted, mesh-shaped ground structures and spacer threads which space the ground structures apart from each other and, in a second step, the spacer threads were severed.

Severing the spacer threads took place immediately following interloop formation at/in the formed-loop knitting machine and the two severed loop-formingly knitted ground structures were wound up separately.

The three-dimensional formed-loop knit was produced using a three-dimensional formed-loop knitting machine having two formed-loop knitting needle beds (front and back needle beds) and five guide bars (guide bar 7, guide bar 6, guide bar 4, guide bar 3 and guide bar 2).

The first textile ground structure was produced using two guide bars, namely guide bar 7 and guide bar 6, on the front needle bed (threaded 1 in/1 out):

Guide bar 7:
(0-1/1-1/2-1/1-1/0-1/1-1/1-2/2-2/3-2/2-2/1-2/2-2/3-2/2-2/2-1/0-0).

Guide bar 6:
(3-2/2-2/1-2/2-2/3-2/2-2/2-1/1-1/0-1/1-1/2-1/1-1/0-1/1-1/1-2/2-2).

The second textile ground structure was produced using two guide bars, namely guide bar 3 and guide bar 2, on the back needle bed (threaded 1 in/1 out):

Guide bar 3:
(2-2/3-2/2-2/1-2/2-2/3-2/2-2/2-1/1-1/0-1/1-1/2-1/1-1/0-1/1-1/1-2).

Guide bar 2:
(1-1/0-1/1-1/2-1/1-1/0-1/1-1/1-2/2-2/3-2/2-2/1-2/2-2/3-2/2-2/2-1).

Guide bar 4 was used to connect the two textile ground structures via spacer threads by binding the spacer threads into every second interloop of the ground structures. The interloops were produced on the front and back needle beds (threaded 1 in/1 out):

Guide bar 4:
(0-1/0-0/0-0/1-0/1-0/0-0/0-0/0-1/0-1/0-0/0-0/1-0/1-0/0-0/0-0/0-1).

Guide bars 7, 6 and 4 were used for the front of the three-dimensional formed-loop knit and guide bars 2, 3 and 4 for the back.

The spacer threads were severed to obtain two medical products each in the form of a fabric having a textile ground structure and thread structures protruding from the textile ground structure. The medical products had altogether three thread plies, two of which formed the textile ground structure and the remaining, the thread structures protruding from the textile ground structure.

### Production Example 3

The medical product was produced in two stages proceeding from polypropylene threads in that, in a first step, a three-dimensional formed-loop knitting machine was used to produce a three-dimensional formed-loop knit having two opposite loop-formingly knitted, mesh-shaped ground structures and spacer threads which space the ground structures apart from each other and, in a second step, the spacer threads were severed.

Severing the spacer threads took place immediately following interloop formation at/in the formed-loop knitting machine and the two severed loop-formingly knitted ground structures were wound up separately.

The three-dimensional formed-loop knit was produced using a three-dimensional formed-loop knitting machine having two formed-loop knitting needle beds (front and back needle beds) and five guide bars (guide bar 7, guide bar 6, guide bar 4, guide bar 3 and guide bar 2).

The first textile ground structure was produced using two guide bars, namely guide bar 7 and guide bar 6, on the front needle bed (threaded 1 in/1 out):

Guide bar 7:
(1-0/1-1/1-2/2-2/2-3/2-2/2-1/1-1)×3.

Guide bar 6:
(2-3/2-2/2-1/1-1/1-0/1-1/1-2/2-2)×3.

The second textile ground structure was produced using two guide bars, namely guide bar 3 and guide bar 4, on the back needle bed (threaded 1 in/1 out):

Guide bar 3:
(2-2/2-3/2-2/2-1/1-1/1-0/1-1/1-2)×3.

Guide bar 2:
(1-1/1-0/1-1/1-2/2-2/2-3/2-2/2-1)×3.

Guide bars 7, 6 and 4 were used for the front of the three-dimensional formed-loop knit and guide bars 2, 3 and 4 for the back.

Guide bar 4:
(1-0/0-0/1-0)×8.

The spacer threads were severed using a hot wire to obtain two medical products each in the form of a textile fabric having a textile ground structure and thread structures protruding from the textile ground structure. Both medical products had in each case three thread plies, two of which formed the textile ground structure and the remaining, the projecting thread structures.

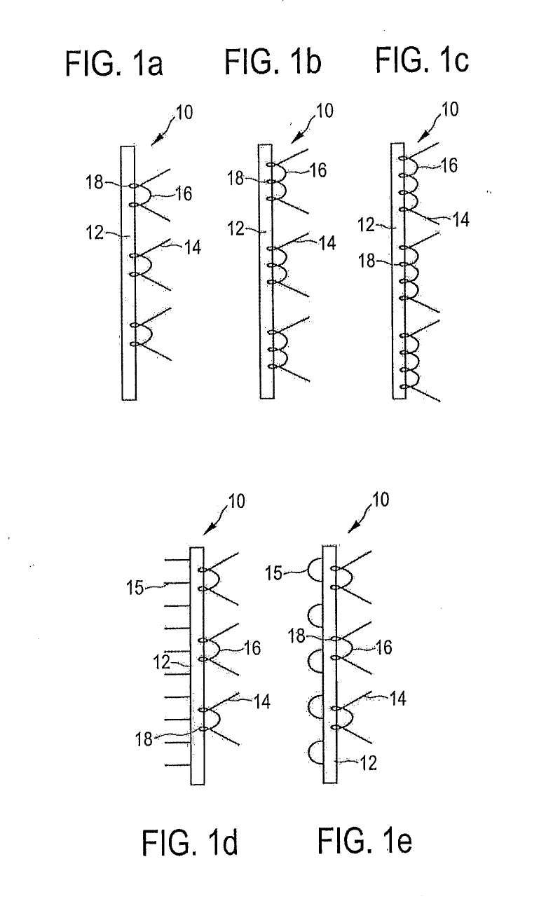

Turning now to the Drawings, FIGS. 1a to e show a schematic depiction of a medical product 10 in the form of a textile fabric having a textile ground structure 12 and thread structures 14 protruding from the textile ground structure 12. The thread structures 14 are each tied pairwise into the textile ground structure 12 in the form of an interloop 16 (FIG. 1a), two interloops 16 (FIG. 1b) or three interloops 16 (FIG. 1c). In other words, the thread structures 14 can have two tie-in points 18 (FIG. 1a), three tie-in points 18 (FIG. 1b) or four tie-in points 18 (FIG. 1c) in the textile ground structure 12 or be, respectively, tied in the textile ground structure 12 two times (FIG. 1a), three times (FIG. 1b) or four times (FIG. 1c).

The thread structures 14 can protrude from one side of the textile ground structure 12 (FIG. 1a).

It may alternatively be provided that the medical product 10 has additional thread structures 15 on an opposite side of the textile ground structure 12 which may be configured as cut pile/tuft threads (FIG. 1d) or as thread loops (FIG. 1e).

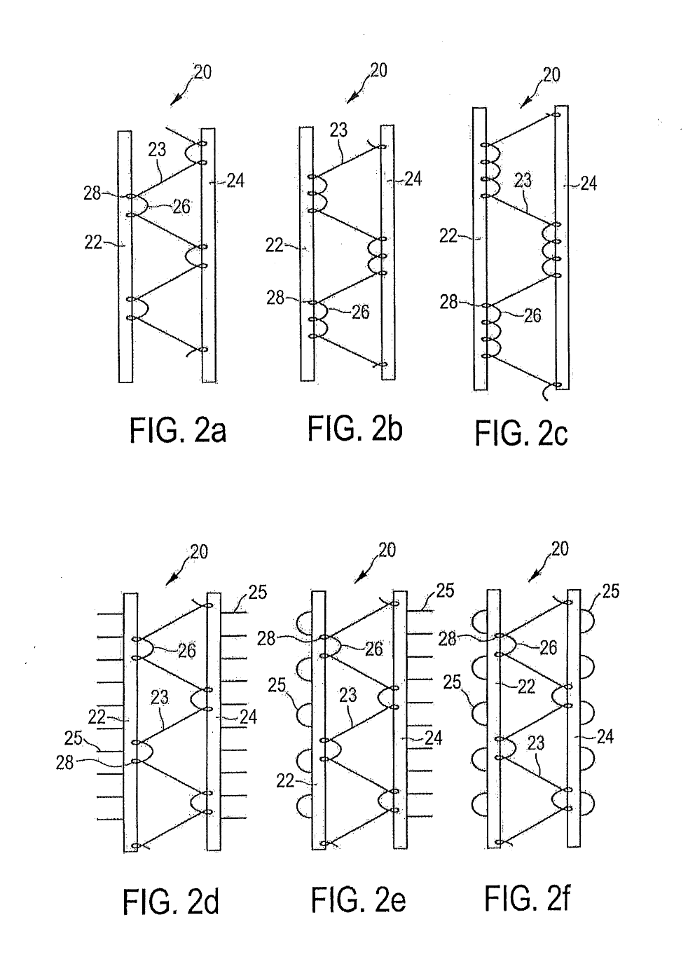

FIGS. 2a to f show a schematic view of a medical product 20 in the form of a spacer fabric having two textile ground structures 22 and 24 which are mutually spaced apart via spacer threads 23.

The spacer threads 23 may be tied into the textile ground structures 22 and 24 in the form of an interloop 26 (FIG. 2a), two interloops 26 (FIG. 2b) or three interloops 26 (FIG. 2c). In other words, the spacer threads 23 can have two tie-in points 28 (FIG. 2a), three tie-in points 28 (FIG. 2b) or four tie-in points 28 (FIG. 2c) in the textile ground structures 22 and 24 or, respectively, be tied into the textile ground structure 22 and 24 two times (FIG. 2a), three times (FIG. 2b) or four times (FIG. 2c).

The textile ground structures 22 and 24 may have additional thread structures 25 protruding from the textile ground structures 22 and 24 in the form of cut pile threads and/or thread loops, for example, on the sides which face away from the spacer threads 23 (FIGS. 2d to f).

Severing the spacer threads 23, for example, with a hot wire or laser, may provide the medical products 10 schematically depicted in FIGS. 1a to 1f.

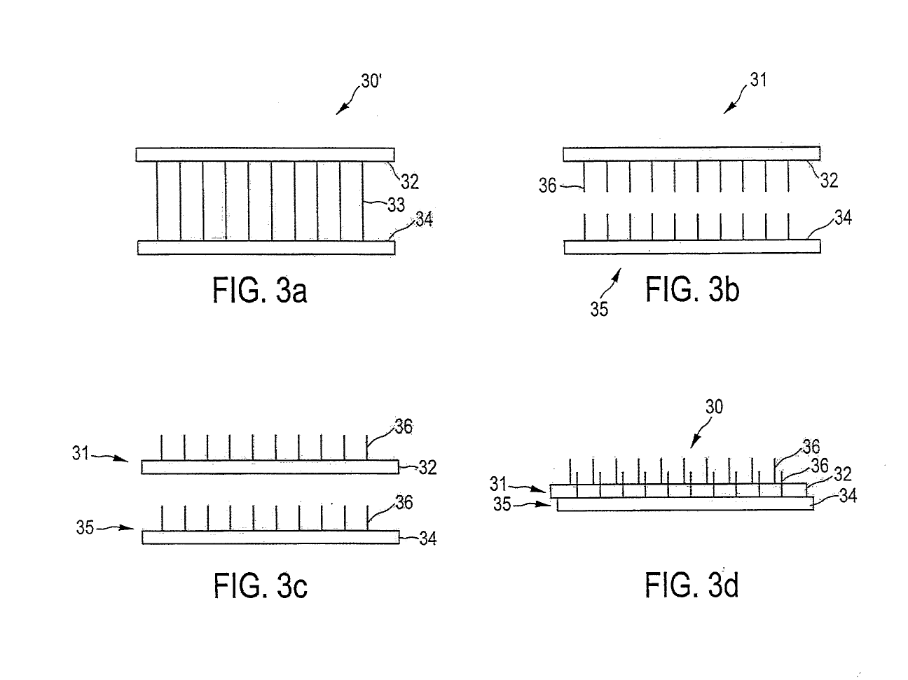

FIGS. 3a to d are a schematic depiction of the production of a medical product 30 (FIG. 1d) proceeding from a spacer fabric 30′ having two opposite textile ground structures 32 and 34 which are mutually spaced apart via spacer threads 33 (FIG. 1a).

The spacer threads 33 of the spacer fabric 30′ are both-sidedly interlooped with the textile ground structures 32 and 34. In this respect, express reference is made to the description hereinabove, especially to the preceding descriptions of figures.

Initially, the spacer threads 33 are cut through to obtain two textile fabrics 31 and 35. The two textile fabrics 31 and 35 each have a textile ground structure 32 or respectively, 34 and also thread structures 36 sticking out therefrom. The thread structures 36 are thus cut-off or -through spacer threads 33. The thread structures 36 protruding from the textile ground structures 32 and 34 are each one-sidedly interlooped with the textile ground structures 32 and 34.

Next, the two textile fabrics 31 and 35 are laid on top of each other such that the thread structures 36 of the bottom textile fabric 35 protrude through interloops, especially pores of the top textile fabric 31.

To secure the two textile fabrics 31 and 35 to each other, the thread structures 36 of the bottom textile fabric 35 or of its ground structure 34 may be connected, especially adhered, thermofixed and/or fused, especially ultrafused, to the ground structure 32 of the top textile fabric 31, for example.

This particular form of connecting the two textile fabrics 31 and 35 together significantly raises, especially doubles, the numbers of protruding thread structures 36 on one side of the product 30. This improves to particular advantage the retaining, especially self-retaining, properties of product 30.

The textile fabric 31 is preferably configured to be non-absorbable. The fabric 31 may, for example, be a polypropylene mesh, in particular a loop-formingly knitted polypropylene mesh. By contrast, the textile fabric 35 is preferably configured to be absorbable. The thread structures 36 are preferably configured to be absorbable.

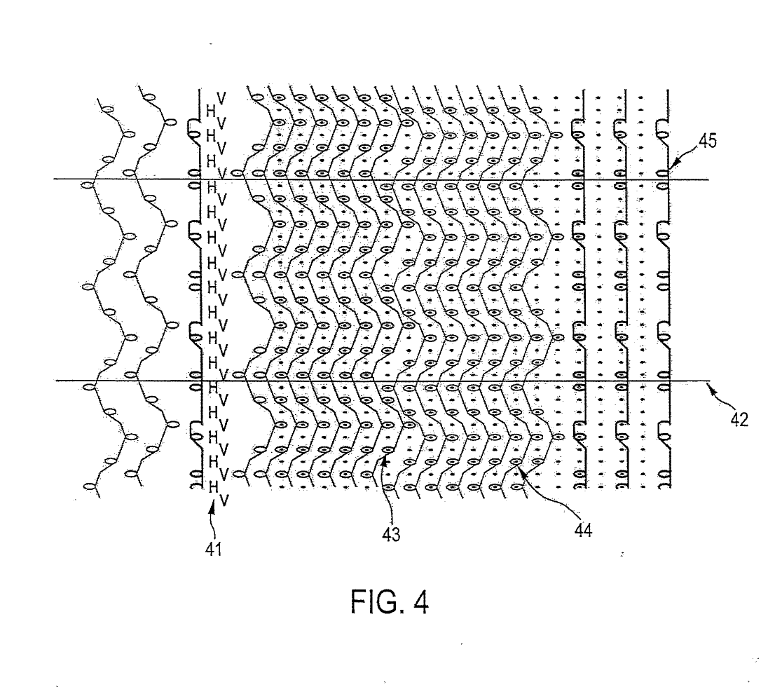

FIG. 4 is a schematic showing the lapping diagram 40 for the medical product produced as per Production Example 1. For clarity, the threads of the individual guide bars are shown side by side and not one above the other.

The reference numerals in FIG. 4 have the following meanings:
Reference numeral 41: needle bed position (V: front and H: back),
Reference numeral 42: repeat line
Reference numeral 43: guide bar 7 (ground structure; front needle bed (fully threaded)),
Reference numeral 44: guide bar 3 (ground structure; back needle bed (fully threaded)) and
Reference numeral 45: guide bar 4 (connecting thread (threaded 1 in, 1 out)).

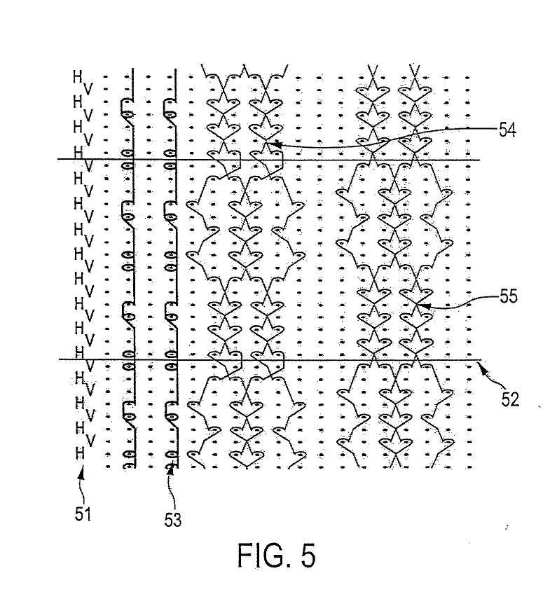

FIG. 5 is a schematic showing the lapping diagram 50 for the medical product produced as per Production Example 2. For clarity, the threads of the individual guide bars are shown side by side and not one above the other.

The reference numerals in FIG. 5 have the following meanings:
Reference numeral 51: needle bed position (V: front and H: back),
Reference numeral 52: repeat line
Reference numeral 53: guide bar 4 (connecting thread (1 in, 1 out)),
Reference numeral 54: guide bars 3 and 2 (ground structure; produced on back needle bed (threaded 1 in, 1 out)) and
Reference numeral 55: guide bars 7 and 6 (ground structure; produced on front needle bed (threaded 1 in, 1 out)).

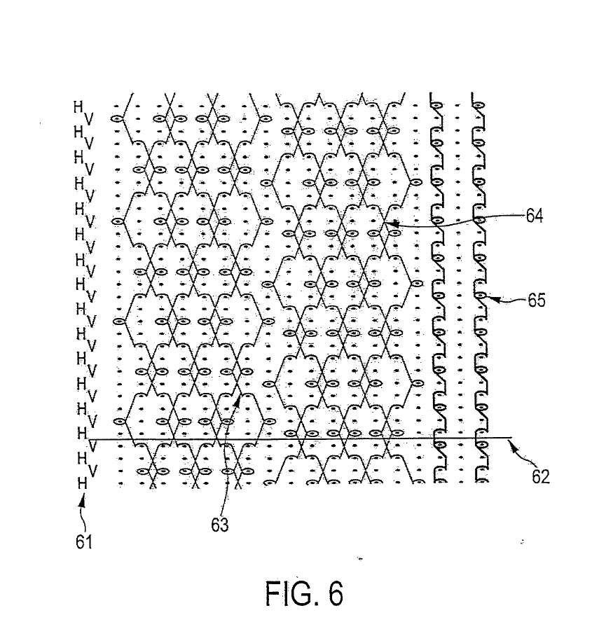

FIG. 6 is a schematic showing the lapping diagram 60 for the medical product produced as per Production Example 3. For clarity, the threads of the individual guide bars are shown side by side and not one above the other.

The reference numerals in FIG. 6 have the following meanings:
Reference numeral 61: needle bed position (V: front and H: back),
Reference numeral 62: repeat line
Reference numeral 63: guide bars 7 and 6 (ground structure; produced on front needle bed (threaded 1 in, 1 out)) and
Reference numeral 64: guide bars 3 and 2 (ground structure; produced on back needle bed (threaded 1 in, 1 out)) and
Reference numeral 65: guide bar 4 (connecting threads (1 in, 1 out).

Although our medical products and processes have been described in connection with specific forms thereof, it will be appreciated that a wide variety of equivalents may be substituted for the specified elements described herein without departing from the spirit and scope of this disclosure as described in the appended claims.

1. A medical product for use in treating/managing hernias, prolapses, urinary incontinence and/or dyspareunia in humans and/or animals comprising at least one textile fabric having a textile ground structure and thread structures which protrude from the textile ground structure, wherein at least some of the thread structures are interlooped with the textile ground structure.

2. The medical product according to claim 1, wherein all thread structures are interlooped with the textile ground structure.

3. The medical product according to claim 1, wherein the thread structures are tied into the textile ground structure, in the form of one interloop and/or in the form of a multiplicity of interloops.

4. The medical product according claim 1, wherein the thread structures are cut threads of a spacer fabric.

5. The medical product according to claim 1, wherein the thread structures are formed on one side of the textile ground structure only and thread loops and/or projecting threads in the form of tuft or pile threads are formed on an opposite side of the textile ground structure.

6. The medical product according to claim 1, wherein the at least one textile fabric is formed of an absorbable polymer selected from the group consisting of polylactide, polyglycolide, poly-ε-caprolactone, poly-para-dioxanone, polytrimethylene carbonate, polyhydroxybutyrate, poly-3-hydroxybutyrate, poly-4-hydroxybutyrate, polyhydroxyvalerate, copolymers, terpolymers thereof, collagen, gelatin, elastin, reticulin, laminin, fibronectin, silk, spider's silk, albumin, fibrin, fibrinogen, starch, amylose, amylopectin, dextran, chitosan, cellulose, alkylcelluloses, methylcellulose, carboxyalkylcelluloses, carboxymethylcellulose, hyaluronic acid, heparin, heparan sulphate, chondroitin sulphate, dermatan sulphate, keratan sulphate, salts thereof and combinations thereof.

7. The medical product according to claim 1, wherein the at least one textile fabric is formed of a non-absorbable polymer selected from the group consisting of polypropylene, polyethylene, high density polyethylene (HDPE), low density polyethylene (LDPE), high molecular weight polyethylene (HMWPE), ultrahigh molecular weight polyethylene (UHMWPE), polyvinylidene dichloride, polyvinylidene difluoride, polytetrafluoroethylene, polyhexafluoropropylene, polytetrafluoropropylene, polyethylene terephthalate, polypropylene terephthalate, polybutylene terephthalate, polyetheretherketone (PEEK), nylon, nylon-6, nylon-6,6, nylon-6,12, polyurethane, copolymers, for example, terpolymers thereof and combinations thereof.

8. The medical product according to claim 1, wherein the thread structures are absorbable.

9. The medical product according to claim 1, wherein the textile ground structure is configured as interlooped fabric.

10. The medical product according to claim 1, wherein the textile ground structure is configured as formed-loop knit mesh.

11. The medical product according to claim 1, which two textile fabrics each have a textile ground structure and thread structures protruding from the textile ground structure.

12. The medical product according to claim 11, wherein the two textile fabrics are in a mutually superposed arrangement wherein at least some of the thread structures of the bottom textile fabric project through interloops of the top textile fabric.

13. A medical product as a spacer fabric with two mutually opposite textile ground structures spaced apart from each other via spacer threads, wherein at least some of the spacer threads are interlooped with at least one of the two textile ground structures.

14. A process for producing one or more medical products according to claim 1, wherein spacer threads of a spacer fabric which space apart two mutually opposite textile ground structures from each other are severed by cutting through to obtain two textile fabrics having a textile ground structure and thread structures protruding from the textile ground structure.

15. The process according to claim 14, wherein the spacer threads are interlooped with at least one of the two textile ground structures to produce the spacer fabric.

16. The process according to claim 14, wherein the spacer threads are tied into at least one of the two textile ground structures in the form of one interloop and/or in the form of a multiplicity of interloops.

17. The process according to claim 14, wherein the spacer fabric is produced using a formed-loop knitting machine with at least two formed-loop knitting needle beds.

18. The process according to claim 14, wherein the spacer threads are severed mechanically, thermally, by a hot wire, ultrasound or a laser.

19. The process according to claim 14, wherein at least one of the two textile ground structures are subjected on a side facing away from the spacer threads or thread structures to the production of thread loops via alternating thread insertions using needles and/or pins.

20. The process according to claim 14, wherein the two textile fabrics obtained are laid on top of each other such that at least some of the thread structures of the bottom textile fabric project through interloops of the top textile fabric.
